(19) United States
(12) Patent Application Publication
(10) Pub. No.: US 2018/0356771 A1
(43) Pub. Date: Dec. 13, 2018

(54) COMPUTER SYSTEM INCORPORATING AN ADAPTIVE MODEL AND METHODS FOR TRAINING THE ADAPTIVE MODEL

(71) Applicant: NANYANG TECHNOLOGYICAL UNIVERSITY, Singapore (SG)

(72) Inventors: Arindam BASU, Singapore (SG); Yi CHEN, Singapore (SG); Subhrajit ROY, Singapore (SG); Enyi YAO, Singapore (SG); Aakash Shantaram PATIL, Singapore (SG)

(21) Appl. No.: 15/761,386

(22) PCT Filed: Sep. 16, 2016
(86) PCT No.: PCT/SG2016/050450

§ 371 Date: Mar. 19, 2018

(30) Foreign Application Priority Data
Sep. 17, 2015 (SG) 10201507753U

(51) Int. Cl. G05B 13/04; G05B 13/02; G06N 3/04; G06N 3/063; A61F 2/72

(52) U.S. Cl. CPC G05B 13/04; G05B 13/027; A61F 2/72; G06N 3/0635; G06N 3/04

(57) ABSTRACT

A computer system is proposed including an adaptive signal processing model of a kind in which a multiplicative section, such as a VLSI integrated circuit, processes data input to the model, using hidden neurons and randomly-set variables, and an adaptive output layer processes the outputs of the multiplicative section using variable parameters. Controllable switching circuitry is proposed to control which data inputs are fed to which hidden neurons, to reduce the number of hidden neurons required and increase the effective number of data inputs. An algorithm is proposed to selectively disable unnecessary hidden neurons. Normalisation, and a winner-take all stage, may be provided at the hidden layer output.

### CROSS-REFERENCE TO RELATED APPLICATIONS

The present application is a filing under 35 U.S.C. 371 as the National Stage of International Application No. PCT/SG2016/050450, filed Sep. 16, 2016, entitled “COMPUTER SYSETEM INCORPORATING AN ADAPTIVE MODEL AND METHODS FOR TRAINING THE ADAPTIVE MODEL,” which claims priority to Singapore Application No. SG 10201507753U filed with the Intellectual Property Office of Singapore on Sep. 17, 2015 and entitled “COMPUTER SYSETEM INCORPORATING AN ADAPTIVE MODEL AND METHODS FOR TRAINING THE ADAPTIVE MODEL,” both of which are incorporated herein by reference in their entirety for all purposes.

### FIELD OF THE INVENTION

The present invention relates to a computer system in which data input is applied to an adaptive model incorporating a multiplicative stage, with the outputs of the multiplicative stage being applied as inputs to an adaptive layer defined by variable parameters. The invention further relates to methods for training the computer system, and operating the trained system. The invention is particularly, but not exclusively, applicable to a computer system in which the multiplicative stage comprises a very-large-scale-integration (VLSI) integrated circuit including a plurality of multiplicative units which are analog circuits, each analog circuit performing multiplicative operations according to inherent tolerances of its components.

### BACKGROUND OF THE INVENTION

With the rapid increase of wireless sensors and the advent of the age of “Internet of Things” and “Big Data Computing”, there is a strong need for low-power machine learning systems that can help reduce the data being generated by intelligently processing it at the source. This not only relieves the user of making sense of all of this data but also reduces power dissipation in transmission making the sensor node run much longer on battery. Data reduction is also a necessity for biomedical implants where it is impossible to transmit all of the generated data wirelessly due to bandwidth constraints of implanted transmitters.

As an example, consider brain machine interface (BMI) based neural prosthesis—an emerging technology for enabling direct control of prosthesis from neural signal of the brain of the paralyzed persons. As shown in FIG. 1, one or a set of micro-electrodes arrays (MEAs) are implanted into cortical tissue of the brain to enable single-unit acquisition (SUA) or multi-unit acquisition (MUA), and the signal is recorded by a neural recording circuit. The recorded neural signal, i.e. sequences of action potential from different neurons around the electrodes, carries the information of motor intention of the subject.

The signal is transmitted out of the subject to a computer where neural signal decoding is performed. Neural signal decoding is a process of extracting the motor intention embedded in the recorded neural signal. The output of neural signal decoding is a control signal. The control signal is used as a command to control the prosthesis, such as a prosthesis arm. Through this process, the subject can move the prosthesis by simply thinking. The subject sees the prosthesis move (creating visual feedback to the brain) and typically also feels it move (creating sensory feedback to the brain).

Next generation neural prosthesis requires one or several miniaturized devices implanted into different regions of the brain cortex, featuring integration of up to a thousand electrodes, both neural recording and sensory feedback, and wireless data and power link to reduce the risk of infection and enable long-term and daily use. The tasks of neural prosthesis are also extended from simple grasp and reach to more sophisticated daily movement of upper limb and locomotive bipedal. A major concern in this vision is the power consumption of the electronics devices in the neural prosthesis. Power consumption of the implanted circuits are highly restricted to prevent tissue damage caused by the heat dissipation of the circuits. Furthermore, implanted devices are predominantly supplied by a small battery or wireless power link, making the power budget even more restricted, assuming a long-term operation of the devices. As the number of electrodes increases, higher channel count makes it a more challenging task, calling for optimization of each functional block as well as system architecture.

Another issue that arises with the increasing number of electrodes is the need to transmit large amount of recorded neural data wirelessly from the implanted circuits to devices external to the patient. This puts a very heavy burden on the implanted device. In a neural recording device with 100 electrodes, for instance, with typical sampling rate at 25 Ksa/s and a resolution of 8 bits, the wireless data rate can be as high as 20 Mb/s. Some methods of data compression are therefore highly desirable. It would be desirable to include a machine learning capability for neural signal decoding on-chip in the implanted circuitry, to provide an effective way of data compression. For example, this might make it possible to transmitted wirelessly out of the subject only the prosthesis command (e.g. which finger to move (5 choices) and in which direction (2 choices) for a total of 10 options, which can be encoded in 4 bits). Even if this is not possible, it might be feasible to wirelessly transmit only some pre-processed data with reduced data rate compared to the recorded neural data.

In the field of BMI, the neural decoding algorithms used are predominantly based on active filtering or statistical analysis. These highly sophisticated decoding algorithms work reasonably well in the experiments but requires significant amount of computation efforts. Therefore, the state-of-the art neural signal decoding are mainly conducted on either a software platform or on a microprocessor outside of the brain, consuming a considerable amount of power, thus making it impractical for the long-term and daily use of the neural prosthesis. As discussed above, the next generation neural prosthesis calls for a miniaturized and less power hungry neural signal decoding that achieves real-time decoding. Integrating the neural decoding algorithm with neural recording devices is also desired to reduce the wireless data transmission rate.

Previous literature [1] has proposed a VLSI random projection network and a machine learning system that uses the VLSI random projection network for input vector projection. The machine learning algorithm used is a two layer neural network called an Extreme Learning Machine (ELM) with random fixed input weights. The VLSI random projection network developed in [1] exploits inherent transistor random mismatch in modern CMOS processes, massive parallelism and programmability of digital circuits, achieving a very power efficient solution to perform multiplication-and-accumulation (MAC) operation.

Referring to FIG. 2, an application of the VLSI random projection network is illustrated. This application is disclosed in [1]. A micro-electrode array (MEA) 1 has been implanted into the brain of a subject. The MEA includes: a unit 2 comprising electrodes for recording of neural signals; a transmitting/receiving (TX/RX) unit 3 for transmitting the neural recordings out of the subject (and optionally receiving control signals and/or power); and a power management unit 4 for controlling the units 2, 3.

The subject also wears a portable external device (PED) 5 comprising: a TX/RX unit 6 for receiving the neural recordings from the unit 3 of the MEA 1; a microcontroller unit (MCU) 7 for pre-processing them, and a machine learning co-processor (MLCP) 8 for processing them as described below. The control output of the MLCP 8 is transmitted by the unit 6 to control a prosthesis 9.

In a second application of the VLSI random projection network, the MLCP 8 is located not in the PED 5 but in the implanted MEA 1 This dramatically reduces the data which the unit 3 has to transmit out of the subject, and thus dramatically reduces the power which has to be provided by the power management unit 4. As described below, certain embodiments of the invention are integrated circuits which are suitable for use as the MLCP in such a scenario.

As depicted in FIG. 3, the ELM algorithm is a two layer neural feed-forward network with L hidden neurons having an activation function g: R→R [1].

The network includes d input neurons with associated values x₁, x₂, . . . , x_{d}, which can also be denoted as a vector x with d components. Thus, d is the dimension of the input to the network.

The outputs of these d input neurons are input to a multiplicative section comprising hidden layer of L hidden neurons having an activation function g: R→R. [4] Without loss of generality, we consider a scalar output in this case. The output o of the network is given by:

o=Σⱼ^{L}βⱼhⱼ=Σⱼ^{L}βⱼg(wⱼ^{T}x+bⱼ),wⱼ,x∈R^{d},bⱼ∈R (1)

Note that in a variation of the embodiment, there are multiple outputs, each having an output which is a scalar product of {hⱼ} with a respective vector of L weights βⱼ. The value (wⱼ^{T}x+bⱼ) may be referred to as the activation yⱼ.

In general, a sigmoidal form of g( ) is assumed though other functions have also been used. Compared to traditional back propagation learning rule that modifies all the weights, in ELM wᵢ and bᵢ are set to random values and only the output weights βⱼ, need to be tuned based on the desired output of N items of training data T=[t₁, tn, . . . t_{N}], where tₙ is the desired output for n-th input vector xⁿ. Therefore, the hidden-layer output matrix H is actually unchanged after initialization of the input weights, reducing the training of this single hidden layer feed-forward neural network into a linear optimization problem of finding a least-square solution of β for Hβ=T, where β is output weights and T is the target of the training.

The desired output weights (variable parameters), are then the solution of the following optimization problem:

\hat{β}=min_{β}∥Hβ−T∥ (2)

where β=[β₁ . . . β_{L}] and T=[t₁ . . . t_{N}]. The ELM algorithm proves that the optimal solution \hat{β} is given by \hat{β}=H^{†}T where H^{†} denotes the Moore Penrose generalized inverse of a matrix.

The output weights can be implemented in digital circuits that facilitate accurate tuning. The fixed random input weights of the hidden neurons, however, can be easily realized by random transistor mismatch, which commonly exists and becomes even profounder in the scaling modern deep sub-micrometer CMOS process. Inspired by this idea, a microchip implementing VLSI “random projection network” is proposed in [1] to realize the fixed random input weights and hidden layer activation of the ELM. The VLSI random projection network microchip can co-operate with a conventional digital processor to form a machine learning system using ELM.

The architecture of the proposed classifier that exploits the d×L random weights of the input layer is shown in FIG. 4. A decoder 10 receives the neural recordings and separates it into d data signals indicative of different sensors. The VLSI random projection network consists of three parts—(a) input handling circuits (IHCs) to convert digital input to analog current, (b) a current mirror synapse array 11 for multiplication of input current with random weight and summing up along columns and (c) a current-controlled-oscillator (CCO) neuron based ADC. Thus, a single hidden neuron comprises a column of analog circuits (each of which is labelled a synapse in FIG. 4, and acts as a multiplicative unit) and a sum unit (the COO and corresponding counter) to generate a sum value which is the activation. The hidden neuron also includes a portion of the functionality of a processing unit (e.g. a digital signal processor) to calculate the output of the hidden neuron from the activation.

If binary data are used as the input of the IHCs, the IHCs directly convert it into the input current for the current mirror synapse array by n-bit DAC. Different pre-processing circuits can be implemented in the IHCs to extract features from various input signals.

In the implementation of the concept in [1], minimum sized transistors are employed to generate random input weights wᵢⱼ, exploiting random transistor mismatch, leading to a log-normal distribution of input weight, determined by:

$${w}_{ij} = {e}^{\frac{Δ Vt}{{U}_{T}}} ,$$

where U_{T} is thermal voltage and dVt is the mismatch of transistor threshold voltage and follows a zero-mean normal distribution in modern CMOS processes.

The CCO neurons which perform the ADC each consist of a neural CCO and a counter. They convert output current from each column of the current mirror synapse array into a digital number, corresponding to hidden layer output of the ELM. The hidden layer output is transmitted out of the microchip for further processing. The circuit diagram of CCO-neuron is presented in FIG. 4. The output of CCO-neuron is a pulse frequency modulated digital signal with frequency proportional to input current Iᵢₙ.

As noted above, a digital signal processor (DSP) is usually provided as an output layer of the ELM computational system. The DSP receives the sum values from the VLSI random projection networks, obtains the corresponding outputs of the hidden layer neurons, and generates final outputs by further processing the data, for instance, passing it through an output stage operation which comprises an adaptive neural network with one or more output neurons, associated with respective variable parameters. The (DSP) thus implements an adaptive network. The adaptive network is trained to perform a computational task. Normally, this is supervised learning in which sets of training input signals are presented to the decoder 10, and the adaptive network is trained to generate corresponding outputs. Once the training is over, the entire computational system (i.e. the portion shown in FIG. 4 plus the DSP) is used to perform useful computational tasks.

Note that the VLSI random projection network of [1] is not the only known implementation of an ELM. Another way of implementing an ELM is for the multiplicative section of the adaptive model (and indeed optionally the entire adaptive model) to be implemented in a digital system by a set of one or more digital processors. The fixed numerical parameters of the hidden neurons may be defined by respective numerical values stored in a memory of the digital system. The numerical values may be randomly-set, such as by a pseudo-random number generator algorithm.

### SUMMARY OF THE INVENTION

The present invention aims to provide a new and useful computer system including an adaptively-trained model, comprising a hidden layer of neurons receiving data inputs, and an output layer which receives the outputs of the hidden layer and performs a function of the outputs of the hidden layers based on variable parameters which are adaptively determined.

The present invention further seeks to provide new and useful methods for training the computer system, and methods for used by the trained computer system to process data.

For some applications of the ELM adaptive model described above, the dimension of the input data is quite large (more than a few thousand data values). For some other applications, the network requires a large number of hidden layer neurons (also more than a few thousands) to achieve the best performance. This poses a big challenge to the hardware implementation. This is true both in the case that the ELM is implemented using the tolerances of electrical components to implement the random numerical parameters of the hidden neurons, and in the case that the random numerical parameters are stored in the memory of a digital system.

For example, if the required input dimension for a given application is d, and the adaptive model requires L hidden layer neurons, conventionally, at least d×L random projections are needed for classification. Each neuron requires d random weights and for each dimension, the neurons require L random numbers. However if the maximum input dimension for the hardware is only k (k<d) and the number of implemented hidden layer neurons is N (N<L), the hardware provides a k×N random projection matrix wᵢⱼ (i=1, 2, . . . , k and j=1, 2, . . . , N), which is smaller than d×L.

A first aspect of the invention proposes in general terms that the input layer of the computer system provide a controllable mapping of the input data values to the hidden neuron inputs, and/or the output layer provides a controllable mapping of the hidden neuron outputs to the neurons of the output layer. This makes it possible to re-use the hidden neurons, so as to increase the effective input dimensionality of the computational system, and/or the effective number of neurons.

A first way of doing this is for input data values to be grouped into a plurality of (normally non-overlapping) sub-sets, and the sub-sets of data values are presented to the hidden neuron layer successively. The respective sets of outputs of the hidden neurons are combined by an output layer of the adaptive model. Specifically, for each sub-set, the outputs of the hidden layers are subject to a respective permutation before inputting them into the output layer, and each output layer neuron forms a sum over the sub-subsets.

Doing this may increase the effective dimensionality of the input to the adaptive model. Note that the different sub-sets of the data values are be input successively, but nevertheless combined to produce a single output (per output neuron of the output layer).

A second way of doing this is, in successive steps, to alter the correspondence between the input data values and the inputs of each hidden neuron. Thus, a given data value is successively input to different inputs of a given hidden neuron. In other words, each data value is successively multiplied by a different corresponding one of the random values associated with the input of the hidden neuron. Each neuron of the output layer of the adaptive model performs a sum over the steps of the outputs of the hidden layer neurons, using a different variable parameter for each hidden layer neuron and for each step. Thus, in each step, a given hidden layer neuron influences each output layer neuron in a different way, and accordingly the effective number of hidden neurons is increased.

Experimentally, it has been found the re-using the hidden neurons in these ways may have little or no detrimental effect on the classification accuracy of the computer system in performing the computational task it is trained to carry out.

A second aspect of the invention—which is principally applicable to the case that the hidden layer of neurons are implemented by analog circuits, as in a VLSI random projection network implementation—proposes in general terms that the outputs of the hidden neurons are normalized, to reduce their variation due to temperature and variations in the power supply. This improves the robustness of the adaptive network to those factors.

The second aspect of the invention is motivated by an observation that, due to current mode MAC operation and the CCO-based ADCs used in the known VLSI random projection network, the hidden layer output for a given set of input data varies with temperature and power supply, leading to degradation of classification performance

A third aspect of the invention proposes in general terms that the outputs of the hidden layers are subject to mutual inhibition in which increasing outputs from any of the hidden layer neurons tend to reduce the outputs of the others. This may be expressed as a “soft winner takes all” stage, before the result is input to the output stage. Optionally, the resulting values below a threshold are not employed by the output layer of the network.

The third aspect of the invention is motivated by the observation that the number of MACs needed in the output stage of a known VLSI projection network can be large if the number of hidden neurons is large. The third aspect of the invention may make it possible to reduce number of computational operations (MACs) needed, and may improve classification performance as well.

The first three aspects of the invention can be implemented by pre-processing or post-processing of the input and output data of the multiplicative section of the adaptive model. In the case that the multiplicative section is implemented as a VLSI random project network, the first three aspects of the invention can be implemented in FPGA and/or by a traditional digital signal processor. The techniques expand the capacity and improve the performance of the VLSI random projection network without changing of the physical design of the VLSI random projection network itself.

The techniques of the first three aspects of the invention may be employed during the training stage of the system (i.e. in which the adaptive output layer is trained), and then during the operation of the trained network, when useful computational tasks are being performed.

A fourth aspect of the invention proposes in general terms selectively disabling hidden neurons based on at least one selection criterion indicative of the hidden neuron being of low importance to the output of the computer system.

This fourth aspect of the invention makes it possible to reduce the power consumption of the computer system, since it reduces the number of MACs in both the input and output stage of the ELM.

In principle, one possible selection criterion could be based on the values of the variable parameters which correspond to the hidden neurons in the output layer of the adaptive model. However, this has several disadvantages, including the disadvantage that the output layer has to be trained before a hidden neuron layer is identified for elimination, and then the output layer may have to be retrained after the hidden layer neuron is eliminated.

Accordingly the fourth aspect of the invention proposes that the selection criterion includes presenting training data items to the computer system, and selecting the hidden neurons based statistical properties of the outputs of the hidden neurons.

One way of doing this is by determining the proportion of the training data items for which the activation (i.e. a sum calculated by a respective sum unit over the data inputs to the hidden neuron of a product of the input data and the respective weights, typically plus a respective constant value for that hidden neuron) is within at least one predetermined range.

For example, the selection criterion may identify hidden neurons for which the absolute value of the activation is less than a threshold for at least a certain proportion of the training examples. This possibility is particularly useful in combination with the third aspect of the invention.

Another way of doing this is by determining the proportion of the training data items for which the activation value differs from that of another neuron (e.g. a neighbouring neuron) by an amount which is within at least one predetermined range.

For example, the selection criterion may be based on a count of the number of training examples for which the activation of the hidden neuron, or the difference between that activation and the activation of a neighbouring hidden neuron, is within each of a plurality of respective ranges defined by thresholds. Hidden neurons are selected for elimination if at least one such count is above a further threshold.

The technique of the fourth aspect of the invention is employed before the training of the output layer. Neurons which are selected for disablement are not used during the training of the output layer, or the operation subsequent operation of the computer system to perform useful computational problems.

It is to be understood that the various aspects of the invention may be combined in a single embodiment. Alternatively, the embodiment may incorporate any one or more of the aspects of the invention.

The various aspects of the invention may be implemented within an ELM. However, as an alternative to an ELM, the present approach can be used in other adaptive signal processing algorithms, such as liquid state machines (LSM) or echo state networks (ESN) as well since they too require random projections of the input. That is, in these networks too, a first layer of the adaptive model employs fixed randomly-set parameters to perform multiplicative operations of the input signals, and the results are summed.

The term “adaptive model” is used in this document to mean a computer-implemented model defined by a plurality of numerical parameters, including at least some which can be modified. The modifiable parameters are set (usually, but not always, iteratively) using training data illustrative of a computational task the adaptive model is to perform.

The present invention may be expressed in terms of a computational system, such as a computational system including at least one integrated circuit comprising the electronic circuits having the random tolerances, or, in the case of the first, second and fourth aspects of the invention, a computational system including one or more digital processors for implementing the adaptive model (in this case, the computational system may be a personal computer (PC) or a server).

The computational system may, for example, be a component of an apparatus for controlling a prosthesis.

Alternatively, the invention may be expressed as a method for training such a computational system, or even as program code (e.g. stored in a non-transitory manner in a tangible data storage device) for automatic performance of the method. It may further be expressed in terms of the computational steps performed by the computational system (e.g. during the training of the adaptive network at the output layer, or after training it)

### BRIEF DESCRIPTION OF THE DRAWINGS

Embodiments of the invention will now be described for the sake of example only with reference to the following figures in which:

FIG. 1 shows schematically the known process of control of a prosthesis;

FIG. 2 shows schematically, a known use of a VLSI random projection network;

FIG. 3 shows the structure of ELM model of FIG. 2;

FIG. 4 shows the structure of a machine-learning co-processor of the ELM model of FIG. 3;

FIG. 5 is a circuit diagram of a neuronal oscillator of the VLSI random projection network of FIG. 2;

FIGS. 6(a)-(c) are composed of FIG. 6(a), which shows an example of input dimension expansion in an embodiment of the invention, FIG. 6(b) which shows circuitry which, in the embodiment, is added between the counters of FIG. 4 and output layer; and

FIG. 6(c) which is a timing circuit;

FIGS. 7(a)-(c) are composed of FIG. 7(a), which shows an example of hidden neuron expansion in an embodiment of the invention, FIG. 7(b) which shows circuitry which is included in the decoder of FIG. 4 in the embodiment, and FIG. 7(c) which is a timing circuit;

FIG. 8 shows an example of how an embodiment performs both input dimension and hidden neuron expansion;

FIG. 9 illustrates how the embodiment may implement the second aspect of the invention;

FIG. 10 is an alternative expression of FIG. 9;

FIGS. 11(a)-(c), which are composed of FIGS. 11(a), (b) and (c), shows experimental results of an embodiment of the method including normalisation;

FIGS. 12(a)-(b), which are composed of FIGS. 12(a) and (b), shows the distribution in the output of the hidden layer neurons, for each of three temperatures;

FIG. 13 shows experimental results from embodiments as shown in FIGS. 9 and 10; and

FIG. 14 shows a known liquid state machine, which can be used in a variant of the embodiment.

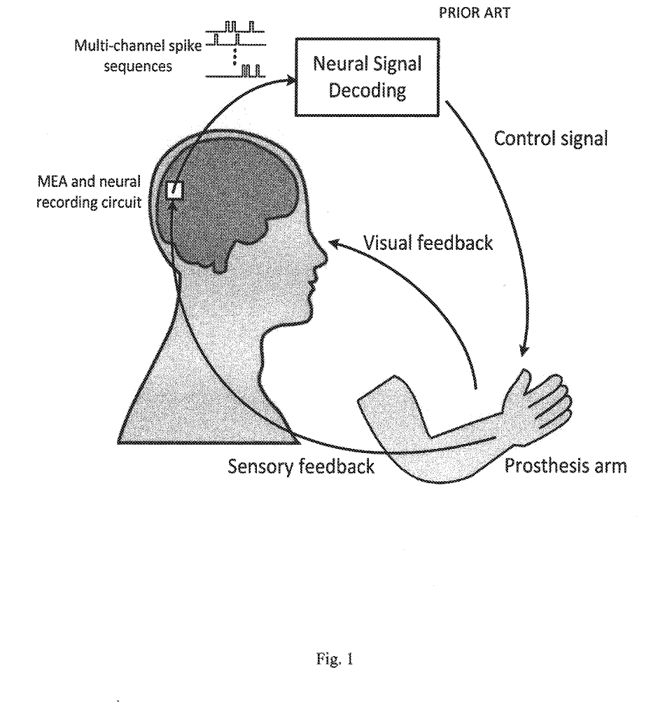

### DETAILED DESCRIPTION OF THE EMBODIMENTS

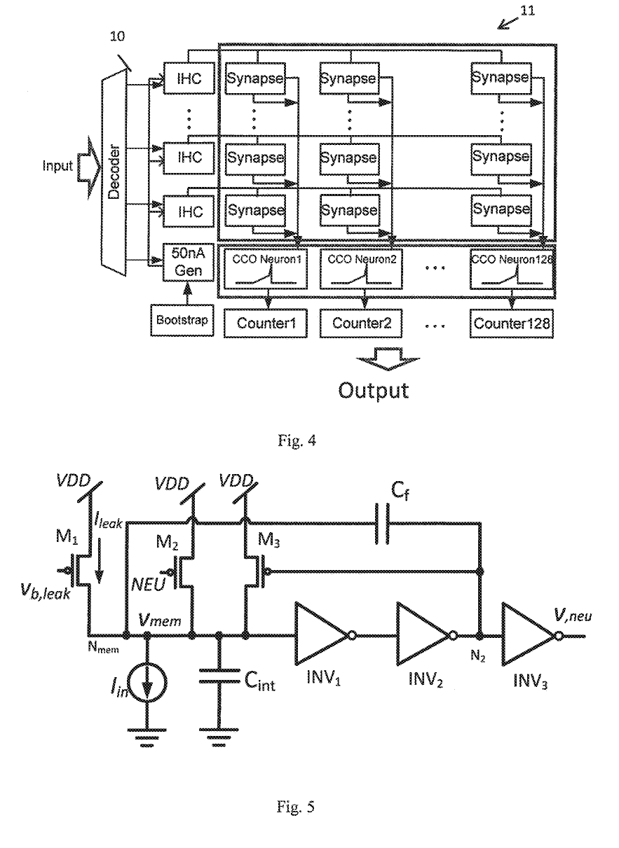

We now describe an embodiment of the invention having various features as described below. The embodiment has the general form illustrated in FIG. 4, but includes four enhanced features, as described below. As described below, other embodiments of the invention may use any combination of these features. Experimental results are supplied from four embodiments which use respective ones of the features.

1. Re-Use of Input Weights

The embodiment has the same overall form as described above for the known VLSI random projection network: that is the structure of FIG. 4 followed by an adaptive output layer which receives the results of the counters. The difference between the embodiment and the known system resides in the construction of the decoder 10, and the interface from the hidden neurons to the output layer. As explained below, these are capable of performing a cyclic permutation. Note that in the experimental results reported below, that cyclic permutation was performed digitally, rather than by the VSLI chip.

In the embodiment we denote the number of neurons of the current mirror synapse array 11 by N, and each neuron includes a corresponding set of k inputs. That is, the number of IHCs is equal to k. Thus, if the decoder 10 and output layer operated as in the known VLSI random projection network, the overall system would have a maximum input dimension of k and be incapable of performing calculations which require more than N hidden neurons.

However, in the embodiment, in fact the decoder 10 receives data which has an input dimension d, where d>k. To expand the effective input dimension from k to d, the decoder 10 divides the input data (a set of d data values) into a plurality of sub-sets of values, where each sub-set includes no more than k data values. The simplest case is that each of these sub-sets has exactly k data values (i.e. d is divisible by k), but if d is not divisible by k (i.e. d=Ak+B where A and B are integers and B is an integer less than k), then the d data values may divided into A sub-sets of k data values and one sub-set of B values. The A+1 subsets can then be handled in the same way as if all the sub-sets comprised k values. The decoder 10 transmits the first sub-set of input data values to the k respective IHCs. The current mirror synapse array 11 multiplies this k dimensional input with the random matrix ωᵢⱼ (i=1, 2, . . . , k and j=1, 2, . . . , N).

Next, the decoder 10 transmits the next sub-set of k input values to the k respective IHCs. However, the decoder 10 also applies a rotation to the N dimensional output. In effect, the random matrix ωᵢⱼ (i=1, 2, . . . , k and j=1, 2, . . . , N) is shifted to ωᵢⱼ=1, 2, . . . , k and j=2, 3, . . . , N, 1). The hidden layer outputs obtained for this sub-set of weights are added respectively to the ones obtained for the first sub-set.

This process continues for successive sub-sets of the d dimensional input data, until the last k-dimensional sub-set of the data. In the last of the d/k subsets the random matrix ωij (i=1, 2, . . . , k and j=1, 2, . . . , N) is shifted to ωij (i=1, 2, . . . , k and j=(d/k), (d/k)+1, . . . , N, 1, 2, . . . , (d/k)−1). More generally, if d is not a multiple of k, then ωij (i=1, 2, . . . , k and j=(ceil(d/k), ceil(d/k)+1, . . . , N, 1, 2, . . . , ceil(d/k)−1), where “ceil (x)” is a function which rounds the argument x up to the next highest integer. Thus, for each hidden neuron, there will be d different random weights.

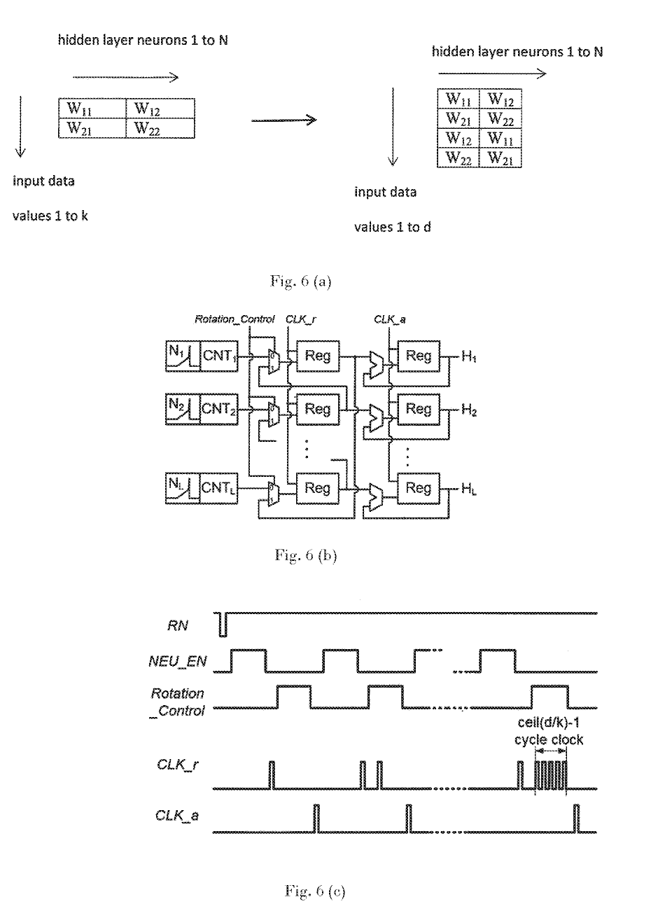

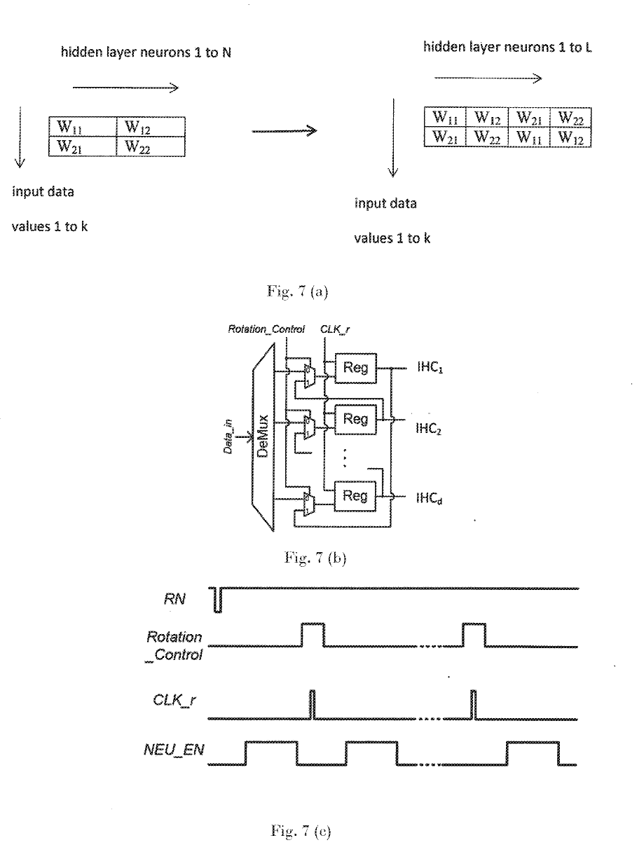

A simple example of this in the case of k=2, N=2 and d=4 is given in FIG. 6(a).

FIG. 6(b) shows a schematic circuit showing how the output layer is modified to produce this effect, and FIG. 6(b) shows the timing diagram.

This method can also be applied to expand the effective number of the hidden layer neurons. Suppose again that the number of data inputs is k and the number of hidden neurons is N. The number of hidden neurons is expanded in ┌L/N┐ steps where the number of projections is increased by N in each step.

In the first step, the outputs for the first L hidden neurons are calculated as in the known VLSI random projection matrix described above.

For the second step, the random matrix ωᵢⱼ (i=1, 2, . . . , k and j=1, 2, . . . , N) is shifted to ωᵢⱼ (i=2, 3, . . . , k, 1 and j=1, 2, . . . , N). Thus, a given one of the k input values is transmitted in the second step to each of the N hidden neurons via a different respective random weight. Thus, in the second step, each of the N hidden neurons is effectively a new hidden neuron.

This process is continued for each of the other ┌L/N┐−2 steps.

This is illustrated in FIG. 7(a). A form of the decoder 10 which is able to achieve this is shown in FIG. 7(b), and its timing diagram is shown in FIG. 7(c).

The output neurons treat the N outputs of the N hidden neurons produced in in each of the ┌L/N┐ steps, as if they had been produced by L hidden neurons. In other words, a given one of the output neurons performs a function in which the N outputs of the hidden layers, for each of the ┌L/N┐ steps, are combined by respective weights (i.e. by N×(L/N)=L weights in total, if L is divisible by N).

Note that the concepts of increasing the effective number of input neurons and the concept of increasing the effective number of hidden neurons can be combined. That is, in each of the ┌L/N┐ steps: (i) the outputs of the hidden layer are calculated successively for each of the (d/k) subsets of neurons, successively permuting the columns of W as explained above, and (ii) the results are added.

Between each of the ┌L/N┐ steps, the rows of the random matrix ωᵢⱼ are permuted. Specifically, after the first step ωᵢⱼ (i=1, 2, . . . , d and j=1, 2, . . . , N) is shifted to ωᵢⱼ (i=2, 3, . . . , d, 1 and j=1, 2, . . . , N), and so on. Thus, in the final step, the random matrix ωij is shifted to (i=ceil(L/N), ceil(L/N)+1, . . . , d, 1, 2, . . . , ceil(L/N)−1 and j=1, 2, . . . , N).

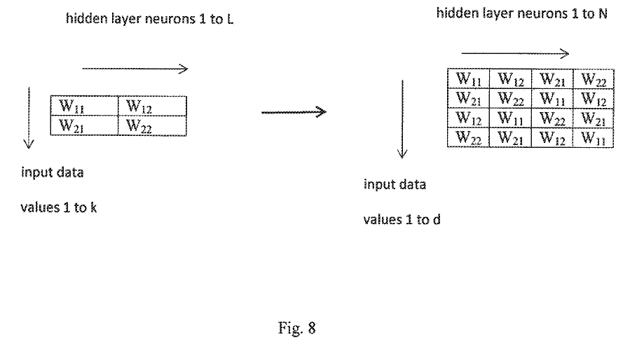

In this way, the maximum input random projection matrix if effectively one having (d*L)×(d*L) weights. An example is given in FIG. 8, where d=L=2, and this produces, in effect, a 4×4 matrix of weights.

2. Normalisation of the Outputs of the Hidden Neurons

Another feature of the embodiment is that the outputs of the random projections are normalized. This reduces variations due to temperature and variability of the power supply, and therefore improves the robustness of VLSI random projection. The normalisation can be performed by a digital processor which performs a second stage multiplication on the respective outputs of each of the hidden layer nodes.

The hidden layer output of the j-th hidden layer node for a certain input vector is denoted by as hⱼ. The normalization conducted here can be expressed by:

$${h}_{j , norm} = \frac{{h}_{j}}{{\sum }_{j = 1}^{L} {h}_{j} / {\sum }_{i = 1}^{D} {x}_{i}} . \qquad ( 3 )$$

The reason for doing this normalization is that the effect of temperature and power supply variation on the hidden layer output can be modelled as multiplication factors in hidden layer output equation and therefore can be eliminated by normalization. The analysis of this point is presented below.

As mentioned before, the hidden layer node (neuron) comprises a CCO that converts input current into a pulse frequency modulated output, and each hidden layer node comprises an output counter that counts number of pulses in the output of CCO in a certain counting window. By analysing the circuit diagram of CCO, as shown in FIG. 5 the output of the j-th hidden layer node can be formulated as:

$${h}_{j} = \frac{{I}_{in , j}}{{C}_{f} \cdot VDD} {t}_{cnt} , \qquad ( 4 )$$

where I_{in,j} is the input current of the j-th hidden layer node, t_{cnt} is length of counting window, and C_{f} and VDD are the capacitance of the feedback capacitor and the voltage output of the power supply of the CCO respectively. I_{in,j}, in turn, is the output current in the j-th column of the current mirror synapse array, and proportional to the strength of input vector [symbol] =[x₁, x₂ . . . x_{D}]. Hence we can model the relation between input vector and hidden layer output as:

hⱼ=Kⱼβ(T,VDD)Σᵢ₌₁^{D}xᵢ, (5)

where, the variation caused by temperature and VDD is modelled by a multiplication term β(T,VDD), and Kⱼ represents the part of path gain from input to j-th hidden layer output that is not affected by temperature and VDD.

Since variation of temperature and VDD is a global effect on the chip scale, we assume

VDD) is the same across different hidden layer nodes. Hence, it can be cancelled by the proposed normalization:

$${h}_{j , norm} = \frac{{h}_{j}}{{\sum }_{j = 1}^{L} {h}_{j} / {\sum }_{i = !}^{D} {x}_{i}} = \frac{{K}_{j} β ( T , VDD ) {\sum }_{i = 1}^{D} {x}_{i}}{{\sum }_{j = 1}^{L} ( {K}_{j} β ( T , VDD ) {\sum }_{i = 1}^{D} {x}_{i} ) / {\sum }_{i = 1}^{D} {x}_{i}} = \frac{β ( T , VDD ) {K}_{j} {\sum }_{i = 1}^{D} {x}_{i}}{β ( T , VDD ) {\sum }_{j = 1}^{L} {K}_{j}} = \frac{{K}_{j}}{{\sum }_{j = 1}^{L} {K}_{j}} {\sum }_{i = 1}^{D} {x}_{i} . \qquad ( 6 )$$

It can be concluded from the deduction that theoretically the proposed normalization can eliminate variation with temperature and power supply. Note that the nonlinear saturation is applied in the digital domain after the normalization. This is done by a subtractor followed by a step of checking the sign bit value.

3. Soft Winner-Takes-all (WTA) Stage

A further feature of the embodiment is a soft WTA stage for processing the hidden layer output before it is passed through the output stage of the known ELM of [1]. The primary difference of the proposed structure in comparison to the known two layer feed-forward ELM architecture is the presence of lateral inhibition in the hidden layer stage.

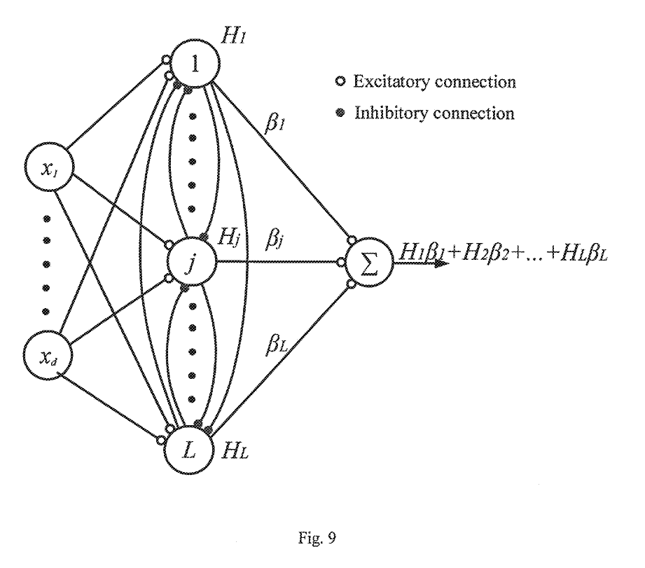

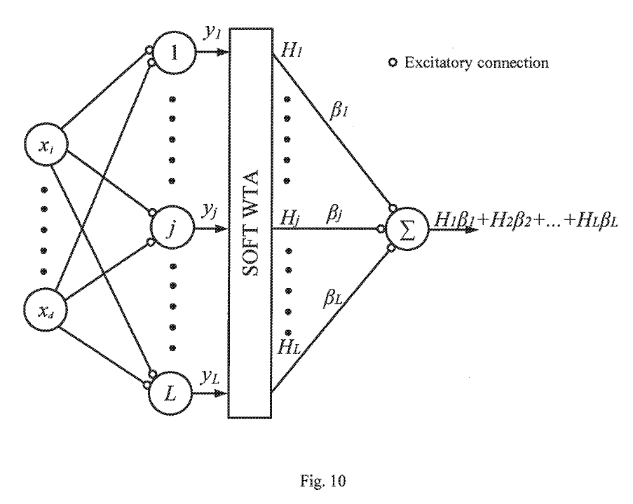

FIG. 9 shows a basic block diagram of the proposed architecture. Depending on its current output each of the hidden layer neurons provides an inhibition signal to all the other neurons. This presence of lateral inhibition can be modeled as a hidden layer without lateral inhibition followed by a soft-WTA gate as depicted in FIG. 10. As explained above, if we consider the weight of the synapse connecting the i-th neuron (i.e. the i-th data input x) to the j-th neuron as given by wⱼᵢ, then yⱼ is given by yⱼ=g(Σ_{d=1}^{d}wⱼᵢxᵢ+bⱼ). The following proposed soft-WTA stage takes [symbol] y₁, . . . , yⱼ, . . . , y_{f} [symbol] as its input and provides an output [symbol] H₁, . . . , Hⱼ, . . . , H_{f} [symbol] where Hⱼ is given by

$${H}_{j} = max ( 0 , {y}_{j} - \frac{1}{L} {\sum }_{j = 1}^{L} {y}_{j} ) .$$

In other words, after calculating the output of hidden layer without inhibition, the embodiment subtracts its mean activation from each hidden neuron output and then subsequently passes it through a linear rectifier unit.

The output of the soft WTA is used in the training phase for tuning output weights βᵢ, and in the operating phase for generating classification output as well.

Since a rectification is applied to the mean-subtracted hidden layer output, the hidden layer nodes with small activation (in other words, a small value for yⱼ) may optionally be suppressed to zero leading to a reduction in the number of MACs which need to be conducted in the following output stage of ELM. Note that for each input vector x, it will typically be a different respective set of the hidden nodes which are turned off.

We will also show in the results section (below) that the proposed soft WTA stage increases classification accuracy compared with the known VLSI random projection network structure without the soft WTA stage.

4. Elimination of Hidden Layer Neurons

A further optional feature of the embodiment is elimination of hidden neurons (i.e. selection of one of more hidden neurons, and then modifying the function performed by the hidden layer such that no output is generated from the selected hidden neurons irrespective of the input vector x). There are various ways in which the hidden neurons may be selected, and the optimal method of selecting hidden layer neurons may vary according to the computational problem the computational system is performing, and the structure of the hidden neurons (e.g. whether they are in one or more layers).

One possible criterion would be to identify which of the hidden neurons j have a low value for \hat{β}ⱼ (that is, the variable value in the output layer corresponding to the j-th hidden neuron).

However, this embodiment uses a criterion for selecting hidden neurons which is dependent on the outputs of hidden neurons for a set of some or all of the training samples xⁿ.

For example, in the manner of the example explained in the previous section, an embodiment may identify, for a given one of the training samples, xⁿ, one of more hidden neurons which have small activation (in other words, a small value for yⱼⁿ). Specifically, a predetermined number of hidden neurons may be identified for which the activation is lowest, or all hidden neurons could be identified for which the activation is below a threshold. This identification could be made for a set of some or all of the training samples. Then, the hidden neurons could be selected based on the proportion of the set of training samples for which the hidden neurons were identified as having low activation. For example, a certain number of hidden neurons may be selected which were identified in this way for the greatest number of training examples.

Alternatively, the present embodiment uses an incognizance check algorithm to select the hidden neurons. The embodiment uses training samples to quantify the “incognizance” for each of the L hidden neurons. In general terms, incognizance for given hidden neuron is given by the proportion of training samples for which it gives an output which is the same. Specifically, the embodiment may determine the proportion of training samples for which the activation of the hidden neuron falls within the same one of a plurality of ranges defined by thresholds, or differs from that of a neighbouring hidden neuron by an amount which is within the same one of a plurality of ranges defined by thresholds. We then sort hidden neurons by this incognizance level and select only the “M” neurons with the least incognizance level.

When the hidden layer is trained, and when the computer system including the hidden layer is in operation for actual test cases, the embodiment power-downs the remaining “L-M” hidden neurons to save energy. Without this aspect of the invention, the energy spend is what is required for D*L MACs in the input stage, L hidden layer non-linearity blocks, L*C MACs in output stage and L*C memory read operations for the output stage weights.

To take one specific example, the embodiment may model input stage weights of ELM as a difference of lognormal distribution. This can be easily realized [2] by finding the pairwise difference of adjacent CCO counts as given by equation (7). For simplicity we use tristate non-linearity given by equation (8).

$$\begin{matrix}{y}_{j}^{′} = {y}_{j} - {y}_{( j + 1 ) mod L} & ( 7 ) \\ {H}_{j} = g ( {y}_{j}^{′} ) = \{ \begin{matrix}- 1 , & {y}_{j}^{′} < - th \\ 0 , & - th \leq {y}_{j}^{′} \leq th \\ + 1 , & th < {y}_{j}^{′}\end{matrix} & ( 8 )\end{matrix}$$

For a given hidden neuron, we calculate the number (cnt1) of training examples for which Hⱼ is equal to −1, the number (cnt2) of training examples for which Hⱼ is equal to 0, and the number (cn3) of training examples for which Hⱼ is equal to +1. The incognizance value for neuron j is then the maximum of cnt1, cnt2 and cnt3.

Based on training samples output for the hidden neurons (for example L=128), we select the “most cognizant” M hidden neurons (i.e. the hidden neurons for which the incognizance value is lowest), and use only these M hidden neurons in the training of the output layer. Also, only these M hidden neurons are used to classify test samples when the computer system is performing useful computing tasks.

Equation (7) may easily be released in a system such as FIG. 4, by finding the differences between the outputs of neighbouring counters. It provides a form of normalisation. The motivation for this normalisation is that in some systems, particularly ones in which the multiplication units are implemented as analog circuits in a VLSI integrated circuit, the weights w₁ may each consist of positive values, and if this is true of the values of x also, then the yⱼ will always be positive. Equation (7) however allows y′ⱼ to take a negative value.

However, in other embodiments the transformation given by equation (7) may be omitted (i.e. y′ⱼ may be replaced in Equation (8) by yⱼ). This may be preferable, for example, in embodiments in which the weights wⱼ include some negative values, so that yⱼ may include negative values. Even in embodiments in which the yⱼ will always be positive, this can be addressed by omitting the normalisation of Equation (7), and instead choosing the three ranges of Hⱼ differently in Equation (8).

Note that there are advantages in selecting hidden neurons using the incognizance method, rather than using \hat{β}ⱼ.

First, to calculate \hat{β}ⱼ one needs to know the variable values in the layer of neurons following that hidden layer. Thus, if there is more than one hidden layer, it will not be possible to eliminate any neurons which are not in the last hidden layer.

Secondly, the pruning based on the activation function can also be used for unsupervised training where there is no labelled output.

Thirdly, the incognizance method is a faster “one-shot” pruning method compared to pruning based on \hat{β}ⱼ which needs \hat{β}ⱼ to be found by iterative solution of equations.

Results

1. Re-Use of Input Weights

Simulation results are shown in Table 1, and indicate the average classification error for an ELM requiring 1000 hidden layer neurons for 50 runs. Each run used a different set of VLSI weights, and thus the experiment shows that the method works for chips with different random values. In this table, the “Error without Weight Rotation” is the known VLSI random projection network described above, where for classification, we have a random matrix for first layer weights with the size of input dimension equal to 1000; the “Error with Weight Rotation” is the embodiment described above, where for classification, the maximum size of the random weight matrix is 128×128. The input weight reuse technique is utilized to expand the random projection matrix by rotation, as described earlier. From this table, we can observe that a similar performance is obtained with the input weight reuse method, which saves hardware resources.

TABLE 1

| | Australian Credit | Leukemia | Bright Data | Adult |
|---|---|---|---|---|
| Input Dimension | 14 | 7129 | 14 | 123 |
| Error without | 13.50 | 17.24 | 1.23 | 15.73 |
| Weight Rotation | | | | |
| Error with | 13.48 | 17.88 | 1.24 | 15.74 |
| Weight Rotation | | | | |

2. Normalisation of the Outputs of the Hidden Neurons

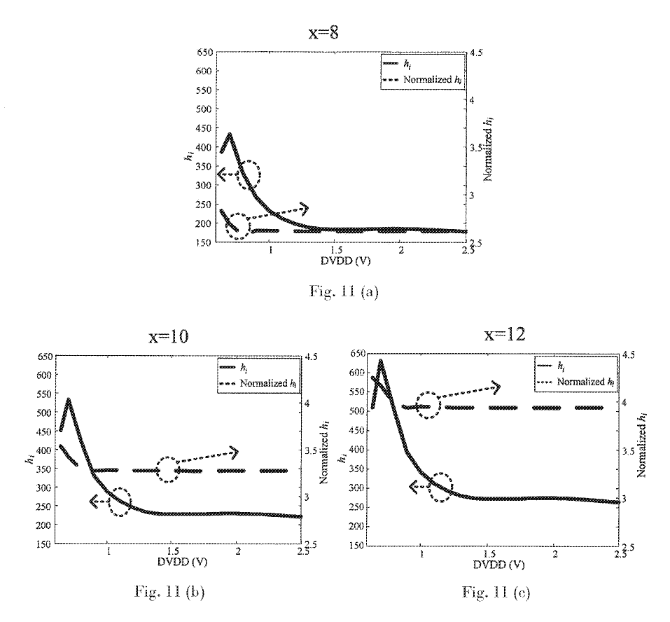

Simulation results are presented here to verify the proposed method of normalization for reducing variation caused by temperature and power supply. The original hidden layer outputs (L=3) are obtained by Cadence simulations where DVDD (the supply voltage to the CCO) sweeps from 0.6 V to 2.5 V and input x (D=1, so there is only one input) successively takes the values 8, 10 and 12. Original and normalized values of one of the hidden layer output are compared in FIG. 11, with different inputs. As can be observed here, the normalized output (in dash lines) varies significantly less due to variation of DVDD than the original output (in solid lines), while the change according to input value is preserved. The circles with arrows highlight which plot refers to the left side y-axis, and which refers to the right side y-axis. The plots of FIGS. 11(a) to (c) show respectively the conventional system, and the normalized system of the embodiment, for a single dimensional input of value 8, 10 and 12. As can be seen, the normalised hidden neuron output has a lesser sensitivity to DVDD.

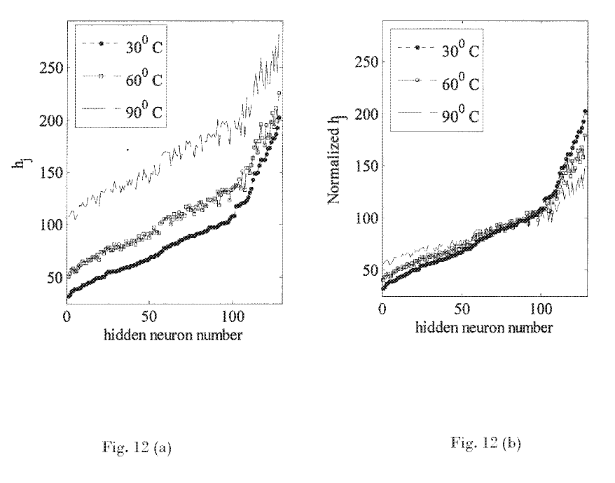

FIG. 12 shows the distribution of the hidden layer outputs for a single dimensional input of value X=8, at each of three temperatures. This is illustrated for the conventional system (FIG. 12(a)) and the normalized system of the embodiment (FIG. 12(b)). As can be seen, the normalized hidden neuron output has lesser sensitivity to temperature.

3. Soft Winner-Takes-all (WTA) Stage

The performance of the embodiment in the case that it includes the soft WTA stage described above is compared to a traditional ELM as in [1]. The experiment is performed using a subset of the widely used MNIST dataset [3]. 600 and 100 images of each handwritten digit (0 to 9) are taken to create the training and the testing set respectively. So, the training set has 6000 images and the testing set has 1000 images. The data from the output of hidden layer without inhibition can be collected from the VLSI chip. On one hand, following the method of [1] this data is directly used to compute the output weights through a pseudo-inverse method. On the other hand, in the embodiment this data is first passed through a soft-WTA and then the output weights are computed by the pseudo-inverse method. The testing accuracy obtained by the traditional method is 85.4% whereas the embodiment obtains 91.8% testing accuracy.

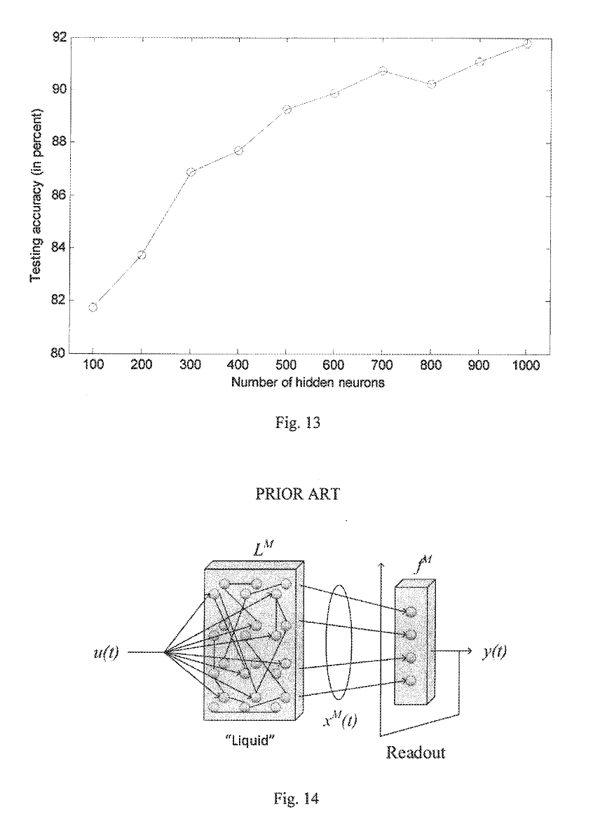

Also, as mentioned earlier, since a large fraction of the neurons are forced to zero for each pattern, the embodiment can reduce the number of MACs in the second layer by eliminating those neurons which have near zero activation for most patterns in the training set For each neuron, we find the percentage of patterns for which the neuron has non-zero activation as a parameter and prune neurons for which this parameter falls below a pre-defined threshold. The performance of the system after different levels of pruning is shown in FIG. 13.

4. Elimination of Hidden Layer Neurons Based on Incognizance

The table below shows average classification error for 100 runs. For sat and vowel, there is standard training and test set and hence for each run we used a different set of weights.

For diabetes and bright there is no fixed distribution of training and test sets and hence for each run we used a different set of weights as well as different training and testing samples (the standard set was always divided into 66% training and 33% test) “Error using all 128 hidden neurons” is the error if we use all available 128 hidden neuron outputs (case 1). “Error using all first M hidden neurons” is the error if we save energy by directly selecting the first M of the 128 hidden neurons (case 2), and as expected this classification error is higher than for case 1. “Error using selective M hidden neurons” is the error in the embodiment, when the method of incognizance checking is used to power down a selected “128-M” hidden neurons. As can be seen from table, the embodiment is able to achieve energy savings similar to Case 2 but without much impact on classification error achievable by Case 1.

| | Sat | Vowel | Diabetes | Bright |
|---|---|---|---|---|
| Error using all 128 | 17.67% | 57.86% | 25.16% | 2.20% |
| hidden neurons | | | | |
| Error using first M | 21.86% | 59.16% | 26.27% | 2.75% |
| hidden neurons | | | | |
| Error using M | 17.84% | 58.34% | 25.33% | 2.18% |
| selected hidden | | | | |
| neurons | | | | |
| L | 128 | 128 | 128 | 128 |
| M | 64 | 96 | 64 | 80 |
| Savings | 50% | 25% | 50% | 37.5% |

Commercial Applications of the Invention

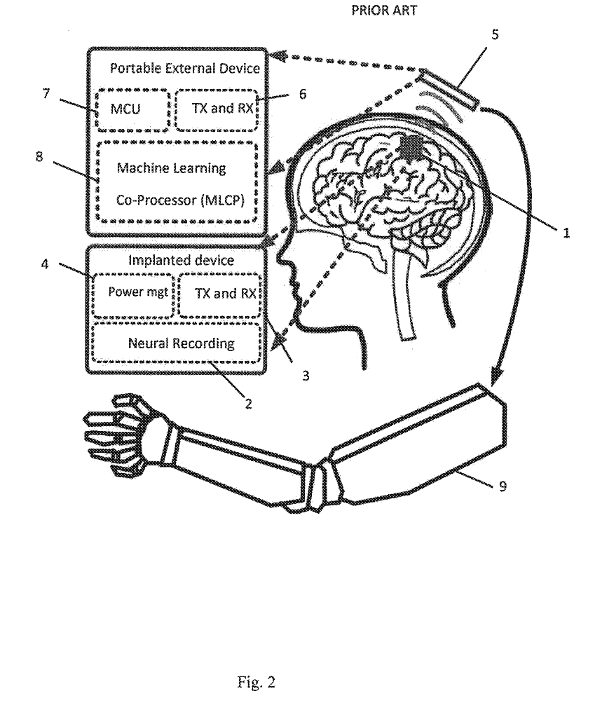

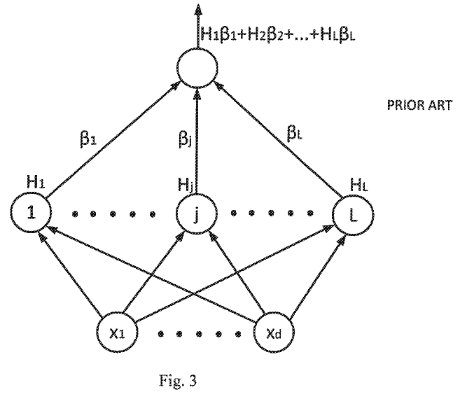

A machine learning system which is an embodiment of the present invention can be used in any application requiring data based decision making in low-power. In particular, embodiments of the invention may be employed in the two applications described above with reference to FIG. 2. Here, we outline several other possible use cases:

1. Implantable/Wearable Medical Devices:

There has been a huge increase in wearable devices that monitor ECG/EKG/Blood Pressure/Glucose level etc. in a bid to promote healthy and affordable life styles. Typically, these devices operate under a limited energy budget with the biggest energy hog being the wireless transmitter. An embodiment of the invention may either eliminate the need for such transmission or drastically reduces the data rate of transmission. As an example of a wearable device, consider a wireless EEG monitor that is worn by epileptic patients to monitor and detect the onset of a seizure. An embodiment of the invention may cut down on wireless transmission by directly detecting seizure onset in the wearable device and triggering a remedial stimulation or alerting a caregiver.

In the realm of implantable devices, we can take the example of a cortical prosthetic aimed at restoring motor function in paralyzed patients or amputees. The amount of power available to such devices is very less and unreliable—being able to decode the motor intentions within the body in a micropower budget enable drastic reduction in data to be transmitted out.

2. Wireless Sensor Networks:

Wireless sensor nodes are used to monitor structural health of buildings and bridges or for collecting data for weather prediction or even in smart homes to intelligently control air conditioning. In all such cases, being able to take decisions on the sensor node through intelligent machine learning will enable long life time of the sensors without requiring a change of batteries. In fact, the power dissipation of the node can reduce sufficiently for energy harvesting to be a viable option. This is also facilitated by the fact that the weights are stored in a non-volatile manner in this architecture.

3. Data Centres:

Today, data centres are becoming more prevalent due to the increasing popularity of cloud based computing. But power bills are the largest recurring cost for a data centre [23]. Hence, low-power machine learning solutions could enable data centres of the future by cutting their energy bills drastically.

### VARIATIONS OF THE INVENTION

A number of variations of the invention are possible within the scope and spirit of the invention, and within the scope of the claims, as will be clear to a skilled reader.

One of these variations is that many of the techniques explained above are applicable to reservoir computing systems, which are closely related to ELMs. In general, a reservoir computing system refers to a time variant dynamical system with two parts—(1) a recurrent connected set of nodes (referred to as the “liquid” of “the reservoir”) with fixed connection weights to which the input is connected and (2) a readout with tunable weights that is trained according to the task. Two major types of reservoir computing systems are popularly used—the Liquid state machine (LSM) and the Echo state network (ESN). FIG. 13 shows a depiction of a LSM network where the input signal u(t) is connected to the “liquid” of the reservoir which implements a function L^{M} on the input to create internal states x^{M}(t), i.e. x^{M}(t)=(L^{M}u)(t). The states of these nodes, xM(t) are used by a trainable readout f^{M}, which is trained to use these states and approximate a target function. The major difference between LSM and ESN is that in LSM, each node is considered to be a spiking neuron that communicates with other nodes only when its local state variable exceeds a threshold and the neuron emits a “spike” whereas in ESN, each node has an analog value and communicates constantly with other nodes. In practice, the communication between nodes for ESN and state updates are made at a fixed discrete time step.

Extreme Learning Machines (ELM) can be considered as a special case of reservoir learning where there are no feedback or recurrent connections within the reservoir. Also, typically the connection between input and hidden nodes is all-to-all in ELM while it may be sparse in LSM or ESN. Finally, the neurons or hidden nodes in ELM have an analog output value and are typically not spiking neurons. However, they may be implemented by using spiking neuronal oscillators followed by counters as shown in the patent draft. It is explained in [1] how an ELM can be implemented using a VLSI integrated circuit, and this applies to the presently disclosed techniques also.

Furthermore, although the embodiments are explained with respect to adaptive models in which the synapses (multiplicative units) are implemented as analog circuits each comprising electrical components, with the random numerical parameters being due to random tolerances in the components, in an alternative the multiplicative section of the adaptive model (and indeed optionally the entire adaptive model) may be implemented by one or more digital processors. The numerical parameters of the corresponding multiplicative units may be defined by respective numerical values stored in a memory. The numerical values may be randomly-set, such as by a pseudo-random number generator algorithm. Particularly in this case, disablement of a hidden neuron may include not only disabling the sum unit of the hidden neuron but also the corresponding multiplicative units.

As noted above, in some embodiments the values of yⱼ will always be positive, because the weights ω₁ and the inputs x may each consist of positive values. Particularly in this case, for all aspects of the invention, the processing unit (e.g. DSP) may transform the activations of the hidden neurons using the Equation (7) before working out the corresponding output of the hidden neuron. Thus, the output layer neurons receive as inputs respective values which are obtained using the sum values from a respective pair of neighbouring hidden neurons. Each output layer neuron still has a variable parameter for each respective sum value, but this variable parameter is applied to a neural output which is obtained by applying the function g to the difference between that respective sum value and the sum value of a neighbouring hidden layer neuron. If this is indeed the function performed by the processing unit, then the use of Equation (7) is particularly suitable in implementing the fourth aspect of the invention.

### REFERENCES

The disclosure of the following references is incorporated herein:
[1] G.-B. Huang, H. Zhou, X. Ding, and R. Zhang, “Extreme learning machine for regression and multiclass classification,” IEEE Trans. Syst., Man, Cybern. B, Cybern., vol. 42, no. 2, pp. 513-529, April 2012.
[2] A. Patil, S. Shen, E. Yao, and A. Basu, “Random projection for spike sorting: Decoding neural signals the neural network way,” in Biomedical Circuits and Systems Conference (BioCAS), 2015 IEEE, pp. 1-4, October 2015. [3] Y. LeCun, L. Bottou, Y. Bengio, and P. Haffner. “Gradient-based learning applied to document recognition.” Proceedings of the IEEE, 86(11):2278-2324, November 1998.
[4] “Compact, Low-power, Machine Learning System utilizing physical device mismatch for classifying binary encoded or pulse frequency encoded digital input with application to neural decoding,” SG patent application no. 10201406665V

1. A computational system to implement an adaptive model to process a plurality of input signals, the system including:
a multiplicative section comprising a plurality of multiplicative units;
an input handling section, for receiving the input signals, and transmitting them to a corresponding plurality of the multiplicative units defined by a first mapping, the multiplicative units being arranged to perform multiplication operations on the corresponding input signals according to respective numerical parameters;
a summation section comprising a plurality of sum units for forming a respective plurality of sum values, each sum value being obtained using the sum of a respective plurality of the results of the multiplication operations defined by a second mapping between multiplicative units and sum units; and
a processing unit for receiving the sum values, and generating an output as a function of the sum values and a respective set of variable parameters;
the system further comprising a control system operative to vary selectively at least one of the first and second mappings.

2. A computational system according to claim 1 in which the input handling system is operative to transmit the data inputs to the multiplicative unit in successive sub-sets, and the control system is operative to control the second mapping to be different for each sub-set, each sum unit of the summation section being operative to sum results of the corresponding multiplication operations for the successive sub-sets of the data input values.

3. A computational system according to claim 1 in which the control system is operative, in each of successive steps, to change the first mapping, the processing unit being operative to generate one or more second sum values, each second sum value being a sum over the steps of the corresponding outputs of a plurality of the sum units weighted with a different variable parameter for each sum unit and each step.

4. A computational system to implement an adaptive model to process a plurality of input signals, the system including:
a multiplicative section comprising a plurality of multiplicative units, each comprising respective electrical components;
an input handling section, for receiving the input signals, and transmitting them to a corresponding plurality of the multiplicative units, the multiplicative units being arranged to perform multiplication operations on the corresponding input signals according to respective numerical parameters;
a summation section comprising a plurality of sum units for forming a respective plurality of sum values, each sum value being obtained using the sum of a respective plurality of the results of the multiplication operations;
a modification layer for modifying the sum values by identifying the sum values which are below a threshold and setting the identified sum values to zero; and
a processing unit for receiving the modified sum values, and generating an output as a function of the modified sum values and a respective set of variable parameters.

5. A computational system according to claim 4 in which the threshold value is formed from an average of the sum values.

6. A computational system to implement an adaptive model to process a plurality of input signals, the system including:
a multiplicative section comprising a plurality of multiplicative units;
an input handling section, for receiving the input signals, and transmitting them to a corresponding plurality of the multiplicative units, whereby the multiplicative units perform multiplication operations on the corresponding input signals according to respective numerical parameters;
a summation section having a plurality of sum units for forming a respective plurality of sum values, each sum value being obtained using the sum of a respective plurality of the results of the multiplication operations;
a processing unit for receiving the sum values, and generating an output as a function of the modified sum values and a respective set of variable parameters;
a selective disablement unit for identifying ones of the sum units for which, over a set of training input signals, the respective outputs of the sum units meet a similarity criterion, and disabling those sum units.

7. A computational system according to claim 6 in which the similarity criterion is based on the number of the set of training input signals for which the sum value of the sum unit is within at least one range defined by at least one threshold.

8. A computational system according to claim 6 in which the similarity criterion is based on the number of the set of training input signals for which the difference between the sum value of the sum unit, and the sum value of another sum unit, is within at least one range defined by at least one threshold.

9. A computational system according to claim 6 in which the similarity criterion is based on the highest of (i) the number of the set of training input signals for which the sum value, or the difference between the sum value and the sum value of another said sum unit, is below a first threshold, (ii) the number of the set of training input signals for which the sum value, or the difference between the sum value and the sum value of another said sum unit, is above the first threshold and below a second threshold higher than the first threshold, or (iii) the number of the set of training input signals for which the sum value, or the difference between the sum value and the sum value of another said sum unit, is above the second threshold.

10. A computational system according to claim 6 in which the criterion is selected to disable a predetermined proportion of the sum units.

11. A computational system according to claim 1 in which the numerical parameters of the corresponding multiplicative units are set randomly.

12. A computer system according to claim 11 in which the multiplicative units are implemented as respective analog circuits, each analog circuit comprising one or more electrical components, the respective numerical parameters being random due to tolerances in the corresponding one or more electrical components.

13. A computational system to implement an adaptive model to process a plurality of input signals, the system including:
a multiplicative section comprising a plurality of analog circuits, each comprising respective electrical components;
an input handling section, for receiving the input signals, and transmitting them to a corresponding plurality of the analog circuits, whereby the analog circuits perform multiplication operations on the corresponding input signals, tolerances in the electrical components causing the multiplication operations to be by respective randomly-set parameters;
a summation section for comprising a plurality of sum units forming a respective plurality of sum values, each sum value being obtained using the sum of a respective plurality of the results of the multiplication operations; and
a processing unit for receiving the sum values, and generating an output as a function of the digital values and a respective set of variable parameters;
the summation section being operative to form a normalisation parameter, and to divide each of the sum values by the normalisation factor.

14. A computational system according to claim 13 in which the normalisation factor is given by Σⱼ₌₀^{L}hⱼ/Σᵢ₌₀^{D}xᵢ, where the values hᵢ are the sum values, the values xᵢ are data input values, the parameter L is the number of analog circuits, the parameter D is indicative of the number of input signals, and the variables j and i are integer variables.

15. A computer-implemented method to process a plurality of input signals, the method including:
(i) receiving the input signals;
(ii) transmitting the input signals to a respective set of multiplicative units defined by a first mapping, the multiplicative units comprising respective electrical components;
(iii) performing multiplication operations on the input signals using the corresponding multiplicative units according to respective numerical parameters;
(iv) forming a plurality of sum values, each sum value being obtained using the sum of a respective plurality of the results of the multiplication operations defined by a second mapping between multiplicative units and sum units; and
(v) generating outputs from respective sub-sets of the sum values defined by a second mapping, each output being a function of the corresponding sum values and a respective plurality of variable parameters;
the method further comprising selectively varying at least one of the first and second mappings.

16-18. (canceled)
